(19) United States
(12) Patent Application Publication
(10) Pub. No.: US 2025/0325609 A1
(43) Pub. Date: Oct. 23, 2025

(54) Method for Tissue Renewal Using A Microalgae-based Biologically Friendly Base Formula To Reverse Disease Progression And Foster Tissue Regeneration In Conditions Of Mucosal Injury And Inflammation In the Mouth And Gastrointestinal Tract

(71) Applicant: OM BIOME, INC., Torrence, CA (US)

(72) Inventor: Julia V. Perederiy, Torrence, CA (US)

(21) Appl. No.: 18/852,463

(22) PCT Filed: Mar. 24, 2023
(86) PCT No.: PCT/US2023/064966

§ 371 Date: Sep. 29, 2024

Related U.S. Application Data
(60) Provisional application No. 63/325,535, filed on Mar. 30, 2022.

(51) Int. Cl. A61K 36/02; A61K 33/06

(52) U.S. Cl. CPC A61K 36/02; A61K 33/06

(57) ABSTRACT

Provided is a composition comprising microalgae and clay, formulated for ingestion, application to skin, an oral cavity, or administration rectally to a mammal. Also provided is a method of treating a mammal. The method comprises administering the above composition to the mammal.

### CROSS REFERENCE TO RELATED APPLICATION

This application claims priority to U.S. Provisional Application No. 63/325,535 entitled “Method For Tissue Renewal Using A Biologically Friendly Base Formula To Restore Balance In The Oral/Digestive Micro-Environment And Reverse Progression Of Inflammatory Diseases Of The Intestinal Track”, filed Mar. 30, 2022, which is herein incorporated by reference.

### FIELD OF THE INVENTION

The present invention is in the technical field of tissue renewal and more particularly to a method for tissue renewal using microalgae-based biologically friendly base formula to reverse progression of disease progression and foster tissue regeneration in conditions of mucosal injury and inflammation in the mouth and gastrointestinal tract.

### BACKGROUND

The human body has an innate capacity to defend against pathogens and rebuild tissues—processes that depend on the following biological elements shown in FIGS. 1 & 2:
1) Immune System—an immediate, highly effective, and regulated response to invading pathogens, which mounts quickly and resolves shortly thereafter;
2) Microbiota—a diverse group of microorganisms serving commensal, symbiotic, or pathogenic functions. These bacteria compete for survival and their composition influences pH, the immune system, and tissue repair processes;
3) Stem Cells—the central component of a self-renewing niche microenvironment responsible for tissue repair and wound healing.

Gradual dysregulation of these biological processes leads to a self-perpetuating cycle of disease. At first, dysregulation is localized, but over time, damage spreads to other areas, organs, and throughout the whole body. This cycle forms the basis of chronic inflammatory disorders, in which symptoms are reflective of the origin of dysregulation. For example, an imbalance of microorganisms—or dysbiosis—may lead to dominance of pathogenic bacteria, which can cause inflammation, reduce turnover of stem cells, and slow tissue repair processes. Likewise, if a short-term defensive inflammatory reaction is not promptly terminated, inflammation can continue spreading to nearby unaffected areas, causing tissue damage and dysbiosis. In turn, chronic inflammation and accumulating oxidative damage can reduce the maximum division potential of proliferating stem cells and thereby disrupt innate processes of healthy aging.

In the mouth and gastrointestinal system, chronic inflammation and dysbiosis can present as periodontal disease, mucositis, bleeding, ulcers, bowel diseases, disrupted stool motility, fissures, pain, reduced nutrient absorption, and/or impaired intestinal barrier function, among other symptoms. These conditions are highly prevalent in modern society, but are also reversible, as long as there is sufficient functional support for the regenerative mechanisms of the human body. Given the right tools, the body can restore its own biological homeostasis (balance).

Microalgae (also known as cyanobacteria or blue-green algae) are one of the earliest life forms on this planet and have nurtured and restored biological ecosystems for millions of years. Microalgae preferably grow in warm, alkaline, nitrogen/phosphorus-rich waters found in Africa, Latin America, and South Asia, as well as in tropical and semi-tropical areas. Known for their high nutritional value and low toxicity, microalgae have been used by ancient cultures as a superfood, by NASA to sustain astronauts in space, and by the United Nations to battle world hunger, as well as additional applications in sustainable agriculture and biofuels. Photosynthetic microalgae have immunomodulating, antioxidant, and antimicrobial/antiviral properties¹, which makes these microorganisms a promising biologically friendly remedy for mucosal inflammation and dysbiosis. As more positive data are published, uses for microalgae are growing, however oral, intestinal, and colorectal applications remain underexplored.

### Oral Cavity:

The oral cavity is a diverse, dynamic, and unique ecosystem that relies on the above-described biological processes to maintain homeostasis and resist toxins and pathogens. The oral microbiota and its propensity for dysbiosis plays a central role in formation of dental plaque (a type of biofilm) and dental caries. Dysbiosis can cause gum inflammation, mouth ulcers, and bad breath. Likewise, dysbiosis can disrupt anchoring of the tooth and its periodontal niche, which can lead to progressive gum disease and tooth loss. Chronic inflammation in the oral cavity also slows division and proliferation of native stem cells that reside below the gum line. These cells populate local surrounding tissues, including the periodontal ligament, which keeps gums in close proximity to the tooth surface and prevents gum recession. Thus, dysbiosis and chronic inflammation lead to bleeding gums, deep periodontal pockets, loose teeth, and gum recession.

Gum disease is painful, immunologically destructive, and widespread, affecting nearly half of US population over age 30, 70% of those over age 65, and more than 750 million people worldwide. Gum disease is one of the most common signs of aging and is clinically associated with more serious conditions like neurodegeneration, heart and kidney disease, and gastrointestinal disturbances, among others. Despite known disease mechanisms, common oral care routines often fail to support biological homeostasis in the oral cavity and can even perpetuate the disease process.

Current practices in oral care reflect a lack of focus on restoration and maintenance of self-regenerating biological ecosystems. Mainstream oral care products like toothpaste and mouthwash are classified as cosmetics and often contain chemicals like triclosan, surfactants, and sweeteners. Such preservatives and foaming agents are masked with taste additives, which may leave a minty-fresh taste but can simultaneously interfere with cellular functions (locally in the oral cavity and systemically throughout the body), including regulation of the immune response, absorption of essential nutrients and minerals, and proliferation of resident stem cells. Likewise, alcohol-based mouthwashes and other antimicrobials (e.g. Listerine, chlorhexidine) kill both pathogenic and beneficial bacteria, which forces a recurring competition for survival within the mucosal biofilm. After each use of an antimicrobial mouthwash, the oral microenvironment attempts to re-establish its natural balance but pathogenic bacteria tend to win, especially if unhealthy lifestyle habits and diet choices persist. The resulting acidic and tartar-prone dysbiosis, alongside spreading inflammation, fuels progressive gum tissue damage, slows healing, and demands frequent repetition of the oral care regimen.

### Gastrointestinal Tract:

The human gastrointestinal (GI) tract and its mucosal lining ensure that the body is protected from external elements and pathogens. The mucosal lining is a series of U-shaped cellular microenvironments much like those found along the gum line. As in the oral cavity, niche microenvironments in the esophagus, stomach, intestines, and the colorectum contain specialized cell types that help with digestion, absorption of nutrients, the immune response to pathogens, and tissue repair. Trillions of beneficial microbes aid in the extraction of energy from food and act as a source of essential nutrients and vitamins. Like in the mouth, composition of bacterial species within the gut mucosa is dynamic and has immunomodulatory properties, functioning in tandem with the immune system to protect against invasion and colonization of pathogens. If pathogenic bacteria dominate, gut mucosal lining can become chronically inflamed, which leads to intestinal pain, bleeding, ulcers, fissures, and general breakdown of protective filtration mechanisms. Tissue repair in the gut is similarly driven by rapidly dividing and undifferentiated stem cells that reside in the base of the U-shaped crypts and give rise to four epithelial lineages that support intestinal function. Dysbiosis and inflammation cause these renewal processes to underperform, which leads to chronic mucosal injury and progressive disease.

Inflammatory gastrointestinal conditions affect more than 40% of people worldwide, with prevalence increasing with advanced age. Symptoms like pain, motility disturbance, visceral hypersensitivity, altered gut microbiota, inflammation, and altered mucosal and immune functions represent a range of conditions like Gastritis, Crohn's disease, Ulcerative Colitis, and Proctitis. These conditions impair quality of life and result in significant healthcare costs. Likewise, these conditions may lead to altered nervous system processing and have been associated with neurodegenerative disease.

Current practices in intestinal and colorectal care are largely focused on controlling the immune response. For example, amino salicylates (e.g. mesalamine) are used to prevent leukocyte recruitment in mild cases, corticosteroids (e.g. prednisone) to dampen inflammation during emergencies, and, in more severe cases, immunomodulatory agents (e.g. mercaptopurine) to inhibit several immune pathways, and biologics/antibodies (e.g. anti-TNFα, anti-interleukins) to neutralize pro-inflammatory proteins made by the immune system. Additional treatments include acetaminophen for mild pain, antibiotics for infections, or antacids to neutralize stomach acid. Unfortunately, many of these drugs have disruptive side effects like abdominal pain, diarrhea, nausea, vomiting, skin reactions, headaches, mood swings, and fatigue, especially if taken for long periods of time. Moreover, immunomodulators and biologics can take as long as 3 months to have a therapeutic effect and require a long-term commitment to treatment. Other remedies for inflammatory conditions of the mouth and gastrointestinal tract include various direct-to-consumer formulations and products that typically have not been evaluated for safety or efficacy.

### Unmet Needs In Oral & Gastrointestinal Mucosal Injury:

Current approaches to chronic inflammatory diseases of the oral and gastrointestinal mucosa do not focus on tissue renewal. Effective approaches must not only terminate the disease process, but strive to rebuild gastrointestinal ecosystems that are naturally resilient and resistant to pathogens. To be effective, novel clinical solutions must incorporate mechanisms to simultaneously (1) curb the inflammatory response, (2) support the competitive advantage of beneficial microbes, and (3) assist proliferation and differentiation of resident stem cells to accelerate mucosal repair, as well as avoid toxic ingredients and unnecessary fillers and taste additives. Microalgae, especially in combination with complementary ingredients, can support these biological functions and help the body heal itself.

Therefore, there is a need for a method for tissue renewal using microalgae in a biologically friendly base formula to reverse progression and foster tissue regeneration in conditions of mucosal injury and inflammation in the mouth and gastrointestinal tract, which overcomes the limitations of prior art.

### BRIEF DESCRIPTION OF THE DRAWINGS

These and other features, aspects, and advantages of the present invention will become better understood with regard to the following description, appended claims, and accompanying figures where:

FIG. 1 illustrates the fundamental principles of mucosal tissue repair according to the present invention, in particular, the conserved U-shaped structure (niche microenvironment) in mucosal membranes in the mouth and along the gastrointestinal tract.

FIG. 2 depicts a summary of microalgae effects on the mechanisms of mucosal tissue repair, as related to immune, microbial, and stem cell functions of niche microenvironments in the mouth and gastrointestinal tract.

FIG. 3 is the composition of one embodiment of a microalgae-based biologically friendly base formula, including complementary therapeutic ingredients: microalgae and clay.

FIG. 4 is a time-lapse of micrographs and statistical analysis of a wound healing experiment using cultured epithelial cells, subjected to a scratch assay and treated with microalgae.

### DETAILED DESCRIPTION OF THE INVENTION

The present invention overcomes the limitations of the prior art by providing a method for tissue renewal using a microalgae-based biologically friendly base formula to reverse progression of disease progression and foster tissue regeneration in conditions of mucosal injury and inflammation in the mouth and gastrointestinal tract. The problems with the prior art are solved by using whole, unaltered, historically safe, and complementary ingredients, including microalgae and clay.

The present invention pertains to pharmaceutical and cosmetic preparations, nutritional and dietary products (including food, supplements, and/or beverage compositions), and methods of use, that are disclosed for use in preventive maintenance and/or restoration of human and veterinary oral and gastrointestinal health, including the oral cavity, intestines, and the colorectum, accomplished through the process of brushing teeth, chewing/dissolving a breath mint/tablet/lollipop/treat, rinsing the mouth/mouthwash, applying a mask/balm/gel to oral tissues, applying as a prophylaxis paste/tooth polish during dental cleaning procedures, flossing, ingesting as a nutritional supplement/timed-release capsule/tablet/gel, rectally inserting a suppository, and/or colorectal irrigating via therapeutic/OTC/prophylactic enema.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any system, any device or part of a system or device disclosed in this disclosure will be determined by its intended use.

Methods and devices that implement the embodiments of the various features of the invention will now be described with reference to the drawings. The drawings and the associated descriptions are provided to illustrate embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises”, and “comprised” are not intended to exclude other additives, components, integers or steps.

In the following description, specific details are given to provide a thorough understanding of the embodiments. However, it will be understood by one of ordinary skill in the art that the embodiments may be practiced without these specific details. Well-known biological processes, circuits, structures, and techniques may not be shown in detail in order not to obscure the embodiments. For example, systems may be shown in block diagrams in order not to obscure the embodiments in unnecessary detail.

Also, it is noted that the embodiments may be described as a process that is depicted as a flowchart, a flow diagram, a structure diagram, or a block diagram. The flowcharts and block diagrams in the figures can illustrate the architecture, functionality, and operation of possible implementations of systems, methods, and computer programs according to various embodiments disclosed. In this regard, each block in the flowchart or block diagrams can represent a module, segment, or portion of code, that can comprise one or more executable instructions for implementing the specified logical function(s). It should also be noted that, in some alternative implementations, the functions noted in the blocks may occur out of the order noted in the figures. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc. When a process corresponds to a function, its termination corresponds to a return of the function to the calling function or the main function. Additionally, each block of the block diagrams and/or flowchart illustration, and combinations of blocks in the block diagrams and/or flowchart illustrations, can be implemented by special purpose hardware-based systems that perform the specified functions or acts, or combinations of special purpose hardware and computer instructions.

Various embodiments provide a method for tissue renewal using a microalgae-based biologically friendly base formula to reverse disease progression and foster tissue regeneration in conditions of mucosal injury and inflammation in the mouth and gastrointestinal tract. The method will now be disclosed in detail.

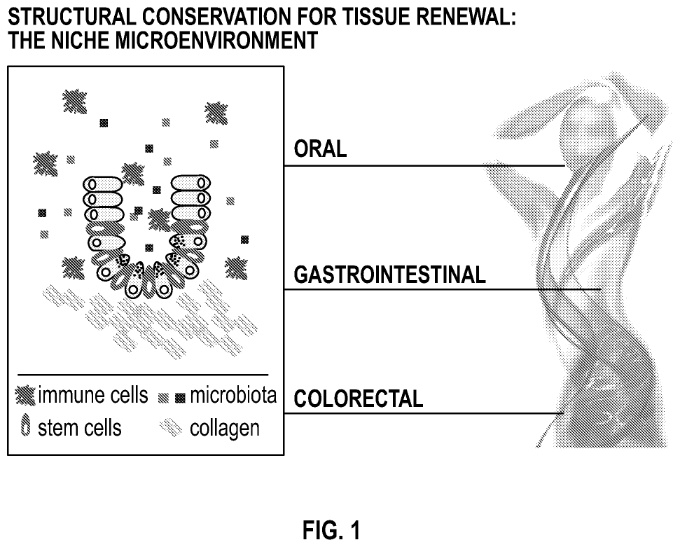

Referring now to FIG. 1, there is shown the structural conservation of the niche microenvironment for tissue renewal, according to the present invention.

As can be seen, the U-shaped dynamic structure is conserved along the entire gastrointestinal system, with variations only in specific cell types and niche depth. Within this structure-anchored to deeper layers by collagenous fibers-three dynamic and interacting forces maintain and renew cellular niche microenvironments: the immune system, microbial diversity, and stem cells.

The immune system is an essential part of the oral and gastrointestinal mucosa, serving as a biological barrier between the body and the external environment. The immune system is responsible for pathogen resistance, as well as maintenance of the internal environment in a state of homeostasis or balance. The protective response mounted by the immune system is dynamic, complex, and highly regulated. If left unchecked, inflammation can spread beyond the first line of defense, causing damage and chronic inflammatory disease. The immune system also interacts with local microorganisms and saliva/GI secretions—a dynamic flow that determines a pro- or anti-inflammatory state within niche microenvironments of the oral and gastrointestinal mucosa.

Outcomes of an ongoing competition between microbes dictates the state of health in the mouth and gastrointestinal tract. Beneficial and pathogenic bacteria compete for survival within a continuous biofilm that lines teeth, cheeks, tongue, esophagus, intestines, and the colorectum. Biofilms are highly structured and spatially organized communities of interacting bacteria or fungi, which adhere to gastrointestinal surfaces and each other to build ecological communities. Microbial communities protect themselves via structural and metabolic reinforcements, secreting compounds to build extracellular polymers and altering their metabolism to render the community impervious to removal.

In case of dysbiosis, pathogenic bacteria dominate the biofilm composition in the mouth/gut, which causes inflammation and slows tissue renewal. Also, pieces of microbial communities can migrate to other areas, which can lead to local and systemic disorders including dental caries and periodontal disease, mucositis, inflammatory gastrointestinal diseases, ulcerative mucosal damage and fissures, diabetes, heart disease, autoimmune conditions, and neurodegeneration. In advanced dysbiosis and infection, first-line clinical treatment is typically antibiotics and antimicrobials, however, these remedies are often ineffective because of the diversity and self-preservation mechanisms of pathogenic biofilms. Efficacy is dependent on the type of antibiotic/antimicrobial chosen, as well as the dose, treatment duration, and level of bacterial resistance. In addition, antibiotics/antimicrobials also kill beneficial bacteria and force the microbe competition to begin anew after the treatment ends, which, in the absence of a biologically friendly intervention, enables dominance of pathogenic bacteria and ongoing dysbiosis. Instead, novel remedies should leverage selective pressure and the dynamic ability of biofilms to undergo microbial population shifts. Such an approach enables a sustainable competitive advantage of beneficial microbes and produces a state that is naturally resistant to pathogens and chronic inflammation and can heal quickly.

Pluripotent stem cells in mucosal niche microenvironments are the building blocks of tissue renewal, however their regenerative capabilities progressively decline over time. In the mouth and gastrointestinal system, these cells reside in a quiescent state in specific locations within the U-shaped anchoring structure and are responsible for self-renewal, as well as maintenance and repair of mucosal surfaces. Stem cells are activated by extrinsic factors generated by other components of the niche microenvironment, including growth factors, morphogenic proteins, biofilm metabolites, and cytokines that regulate proliferation, migration, lineage fate, and differentiation. Chronic inflammation and dysbiosis slow stem cell turnover, which in turn slows the body's ability to repair damaged tissue and heal from injury. However, given the right molecular support, endogenous stem cells in the mouth and gastrointestinal mucosa drive healing from conditions like periodontal disease, oral mucositis, mucosal ulcers, fissures, chronic inflammation, and dysbiosis. Activation, migration, and differentiation of endogenous stem cells leads to reattachment of deep periodontal pockets, regrowth of receding gums and bone, closure of ulcers and fissures, and restoration of gastrointestinal function. Thus, effective remedies for chronic inflammatory disease and dysbiosis must also help maintain homeostatic regulation of endogenous stem cells, which allows the cells to undergo self-renewal and simultaneously support ongoing tissue regeneration.

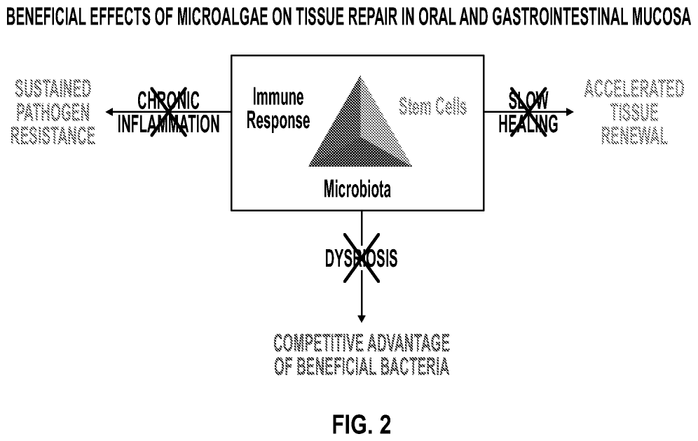

Referring now to FIG. 2, which depicts a summary of positive effects of microalgae on the immune, microbial, and stem cell components of the tissue renewal process, as applies to mucosal tissue repair in the mouth and gastrointestinal tract using the microalgae-based biologically friendly base formula for tissue renewal disclosed herein.

Microalgae (also known as cyanobacteria or blue-green algae) are a diverse group of microorganisms well-recognized for their high nutritional value, low toxicity, and general ability to nurture and sustain life on this planet. Microalgae are described herein as a key ingredient in a biologically friendly base formula to aid tissue renewal, as it pertains to chronic inflammation, dysbiosis, and stem cells. For example, various species of microalgae have anti-inflammatory effects: they reduce cytokine production in allergic rhinitis patients, enhance function in macrophages, and protect against hepatic inflammation in the aged. Likewise, microalgae play a prebiotic role and stimulate growth of probiotics, and can modulate gut microbiota via their antimicrobial and bacteriostatic properties. These features suggest that microalgae can impose selective pressure in oral and gastrointestinal biofilms and induce a dynamic microbial population shift away from dysbiosis toward sustained competitive advantage of beneficial bacteria.

Microalgae contain and secrete bioactive compounds like protein, fatty acids, vitamins, minerals, and essential amino acids, which can be used to replenish cellular resources depleted by chronic inflammation and dysbiosis. Administered as a live microalgal culture, biomass, fraction, extract, and/or isolate, microalgae deliver essential nutrients that support stem cell activity (e.g. proliferation, mobilization, differentiation) and enable regenerative processes of niche microenvironments in mucosal tissue. Nutrients supplied by microalgae nurture cellular ecosystems, which helps terminate the disease process and accelerate tissue renewal, as shown by known positive outcomes in reduction of dental plaque, prevention of gastric ulcers, constipation, diabetes, hypertension, and anemia. However, widespread use of microalgae in healthcare remains limited, in part because of constraints surrounding production, supply chain, and shelf-life.

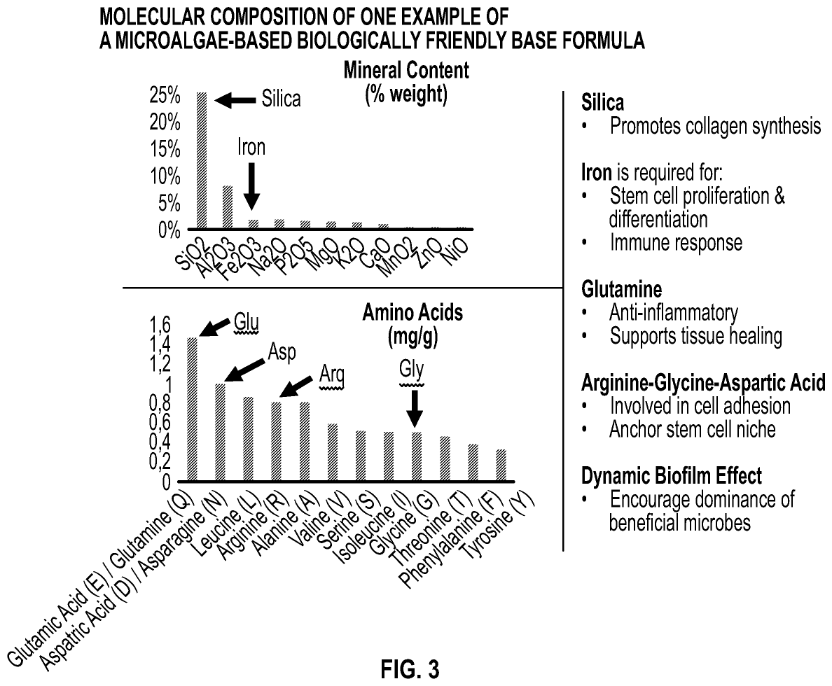

Referring now to FIG. 3, there is shown a molecular composition of one example of a microalgae-based biologically friendly base formula, which contains complementary therapeutic ingredients: microalgae and clay.

Chemical compositions of microalgae and clays vary depending on the geographical source and growing/harvesting conditions. Microalgae typically contain high levels of protein (^{˜}375 g/kg dw), fiber (^{˜}315 g/kg dw), lipids (^{˜}300 g/kg dw), and carbohydrates (^{˜}250 g/kg dw). Likewise, microalgae contain antioxidants, essential amino acids, carotenoids, phenolic compounds (e.g. tocopherols, flavonoids, phenolic acids, tannins, lignans, coumarins), and microelements including iron, zinc, potassium, sodium, calcium, magnesium, manganese, and selenium.

Clay—a complementary therapeutic ingredient to microalgae—refers to a group of microscopic/colloidal crystals organized into layered structures that vary in lattice formation or in types of exchangeable ions. Clay is typically formed in areas with concentrated electrolytes and high pH (^{˜}9), which makes this material an excellent tool to nurture damaged and depleted biological tissues. Likewise, clay's alkalinity helps to temper the acidity that accompanies inflammatory conditions of the mouth and gastrointestinal tract. Microalgae also need high pH for optimal microalgae growth, which makes clay an ideal complement for microalgae formulations. Clay also has a high mineral content and can include montmorillonite or kaolinite, as well as calcium, zinc, potassium, and magnesium, among many others. Functionally, clay is poly-cationic, which aids absorption and excretion of toxins, pesticides and other, as well as metals like lead and copper-all known to accumulate in and cause harm to the human body. Like microalgae, clay also has antibacterial properties and has been suggested for use in cases of diarrhea and antibiotic resistance.

The microalgae-based biologically friendly base formula for tissue renewal contains whole, unaltered, and complementary ingredients with excellent safety profiles and long history of use by ancient cultures and modern nutritionists. The molecular composition of one embodiment of the disclosed base formula includes silica, which promotes collagen synthesis, iron, which is required for the immune response and stem cell proliferation/differentiation, glutamine, which is a potent anti-inflammatory agent and supports tissue renewal, the arginine-glycine-aspartic acid complex, which is involved in cell adhesion and anchors the stem cell niche, and many other essential nutrients and minerals. These minerals are essential for tissue renewal in the human body, including absorbing toxins, rebuilding tooth enamel, and aiding in tissue repair, digestion of food, and absorption of nutrients in the intestine.

Microalgae in the biologically friendly base formula for tissue renewal also have biofilm properties, which can exert a dynamic and positive effect on the competitive biofilm microenvironments in oral and gastrointestinal mucosa. Microalgae support microbial population shifts toward dominance of beneficial bacteria, which leads to sustainable oral/intestinal/colorectal ecosystems that are naturally resistant to pathogens, even with minimal intervention.

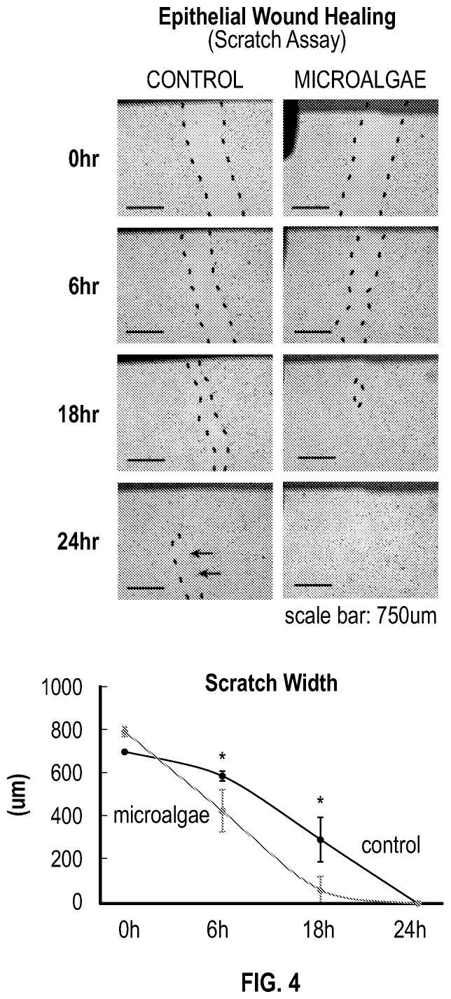

Referring now to FIG. 4, there is shown a time-lapse micrograph and statistical analysis of a wound healing experiment using cultured epithelial cells. Epithelial cells were subjected to a scratch assay and treated with microalgae extract, which accelerated wound closure and reduced scarring.

Basic elements of epithelial/mucosal wound healing and tissue renewal can be modeled using an in-vitro scratch assay, in which cells are seeded in a culture well-plate, grown to confluency, and scratched using a 200 μl pipette tip. After washing the cells to remove debris, wound closure is assessed at regular intervals post-scratch, until cells are again confluent. In the described experiments, Human Small Airway Epithelial Cells were used to conduct the scratch assay. Growth medium with or without microalgae extract was added after the scratch. The setup was monitored periodically until the wound closed. In the Microalgae condition, the scratch closed within 18 hours, compared with the Control condition, in which the scratch was still visible after 24 hours. Likewise, fibrous bundles were observed in Control wells and not in Microalgae wells, suggesting that microalgae may help reduce scarring during the tissue renewal process.

The combination of microalgae and clay delivers complementary tissue renewal effects to reverse disease progression and foster tissue renewal in conditions of mucosal injury and inflammation in the mouth and gastrointestinal tract.

As will be understood by those with skill in the art with reference to this disclosure, there are many delivery vehicles that can be employed, such as, for example:
powder mixture (direct application or dissolved in liquid)
chewable tablets
paste and/or prophylaxis (“prophy”) paste
breath mints/refreshers
gel/balm/foam/cream
polish/scrub
dispensed via applicator/mouth guard/dental sandblaster
chewing sticks/treats
dissolvable strips/wafers
liquid rinse/spray
lollipops/hard candies/lozenges
gummies/jelly candies
dipped/covered floss
orally/rectally administered via solid dosage (e.g. dissolvable tablet, capsule)
orally/rectally administered via liquid dosage (e.g powder dissolved in water, drops)
orally/rectally/skin administered via semi-solid dosage (e.g. gel, foam, balm, mask, cream)
suppository
dispensed via colonic irrigation
enema (therapeutic, prophylactic, in-home, or clinician-administered)
protein bars/cookies/popcorn/chips
smoothie booster/bomb
popsicles/frozen juice bars/ices/flavored sherbets
food booster/additive/supplement

Also, other complementary ingredients may be added, such as, for example:
plants, herbs, extracts, fractions, and derivatives such as anise, fennel, licorice, orange, orange peel, mint, myrrh, neem, rosemary, eucalyptus, echinacea, goldenseal, grape root, propolis, sage, white oak bark, plantain, cinnamon, cloves, turmeric, red thyme, lavender, amla, bilberry fruit, hawthorn berry, mango, black catechu tree, arjuna tree, fever nut, milkweed, horsetail, amla, camphor, rose geranium, calendula, bergamot, cardamom, coconut;
nutrients, such as zinc, sodium, chloride, citrate, calcium, magnesium, folate, bismuth, vitamins A/B/C/D/E/K2, iron, selenium, manganese, silica, potassium, sulfur, copper, choline, fatty acids, and others;
sweeteners, such as honey, syrup, chocolate, molasses, fruit, cane sugar, saccharin, sucralose, xylitol;
other, such as prebiotics, probiotics, menthol, collagen, hydroxyapatite, fluoride, xanthan gum, dicalcium phosphate, magnesium stearate, oils, beets, flaxseed, oats, vegetable glycerin, cellulose, gelatin, pea protein, lecithin, mushrooms, fungal mycelium, cannabinoids, glucosamine, chondroitin, aloe vera, wax, resin, balsam, sap, fat, beeswax, ozone.

### Oral Care:

The method comprises applying a powder to a wet toothbrush or other oral care tool and then using as a medium to brush teeth;

Alternatively, chewing a tablet to mechanically crush the tablet into powder inside the mouth to use as a medium to brush teeth, using a toothbrush or other oral care tool;

Additionally, applying a paste/gel/balm/foam/dissolvable strips/wafers directly onto the gums/tongue/cheeks and/or via dispenser/applicator/mouthguard, and leaving in place as a mask for a period of time;

Moreover, applied by a dental professional as a prophylaxis paste, via a sandblaster, or polish/scrub to clean the tooth surface and remove decay, as used during a routine hygiene appointment (scale and polish) or dental restoration procedures;

Likewise, chewing or sucking on a tablet, lozenge, hard candy, jelly or gummy candy, lollipop, breath mint, stick or treat, used as a daily nutritional boost, therapeutic, and/or breath refresher;

Similarly, dissolving powder mixture in liquid and using as a mouthwash or spraying a liquid version of the invention to rinse mouth and/or swallow as nutritional supplement;

Finally, inserting floss covered with the invention between teeth and gums to deliver nutrients into harder-to-reach/deeper mucosal spaces.

### Gastrointestinal & Colorectal Care:

The method comprises delivering a liquid dosage form of the invention (e.g. powder dissolved in water) via therapeutic, prophylactic, or nutritional enema, administered by a physician, care provider, or individual in a home or professional setting using disposable enema bottles, reusable enema kits, or via colonic irrigation/hydrotherapy devices;

Alternatively, inserting a solid dosage of the invention into the anal opening, via suppository, capsule, or tablet for gradual dissolution and absorption;

Additionally, delivering a semi-solid dosage form of the invention to the skin, anus, rectum, and/or colon using a gel, foam, balm, mask or cream;

Likewise, administering orally and swallowing a liquid, tablet, or capsule, with or without a time release feature;

Finally, eating snacks and treats made with or coated with the invention, including protein bars, cookies, popsicles, frozen bars/ices/flavored sherbets, popcorn, chips, smoothie booster/bomb, as a food additive/nutritional supplement.

### Veterinary Health/Oral, Gastrointestinal, & Colorectal Care:

As will be understood, other mammals that are biologically similar to humans can also benefit from the present invention. There are various ways for the invention to be delivered to non-human users:

As an ingredient in existing pet products and feed;

Application of a powder to a wet toothbrush or other oral care tool and then using as a medium to brush teeth;

Alternatively, via a chewable stick, paste or treat to be chewed and swallowed by the animal as a daily nutritional boost, therapeutic, and/or breath refresher;

Additionally, applying a paste/gel/balm/foam/dissolvable strips/wafers directly onto the gums/tongue/cheeks and/or via dispenser/applicator, and leaving in place as a mask for a period of time;

Moreover, applied by a veterinary professional as a prophylaxis paste, via a sandblaster, or polish/scrub to clean the tooth surface and remove decay, as used during a routine veterinary oral hygiene appointment (scale and polish) or dental restoration procedures;

Likewise, ingested by the animal via administration of capsule or tablet;

Alternatively, insertion of a solid dosage of the invention into the anal opening, via suppository, capsule, or tablet for gradual dissolution and absorption;

Likewise, delivered as a semi-solid dosage form of the invention to the skin, anus, rectum, and/or colon using a gel, foam, balm, mask or cream;

Finally, applied as a liquid enema delivered into the colorectum using a disposable enema bottle, reusable enema setup, or veterinary colonic irrigation/hydrotherapy devices.

What has been described is a new and improved approach as a method for tissue renewal using a microalgae-based biologically friendly base formula to reverse disease progression and foster tissue regeneration in conditions of mucosal injury and inflammation in the mouth and gastrointestinal tract, overcoming the limitations and disadvantages inherent in the related art.

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative feature serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

What is claimed is:

1. A composition comprising microalgae and clay, formulated for ingestion, application to skin, an oral cavity, or administration rectally to a mammal.

2. The composition of claim 1, wherein the composition (a) improves oral health in the mammal when administered to the mammal's oral cavity, and/or (b) improves digestive health in the mammal when ingested by the mammal, and/or (c) improves colorectal health in the mammal when administered to the mammal's colorectal cavity.

3. The composition of claim 1, wherein the composition promotes tissue renewal, cell growth, and/or microbial homeostasis in the mouth and/or gastrointestinal tract in the mammal.

4. The composition of claim 1, wherein the microalgae:clay ratio is 1:1000, 1:500, 1:100, 1:50, 1:10, 1:5, 1:2, 1:1, 2:1, 5:1, 10:1, 50:1, 100:1, 500:1, 1000:1 or any ratio in between.

5. The composition of claim 1, wherein the microalgae:clay ratio is 1:1 to 2:1.

6. The composition of claim 5, formulated for oral administration, wherein the composition is more palatable than a composition outside of that ratio.

7. The composition of claim 1, further comprising a plant or an extract, fraction or derivative thereof.

8. The composition of claim 7, wherein the plant, extract fraction or derivative is anise, fennel, licorice, orange, orange peel, mint, myrrh, neem, rosemary, eucalyptus, echinacea, goldenseal, grape root, propolis, sage, white oak bark, plantain, cinnamon, cloves, turmeric, red thyme, lavender, amla, bilberry fruit, hawthorn berry, mango, black catechu tree, arjuna tree, fever nut, milkweed, horsetail, amla, camphor, rose geranium, calendula, bergamot, cardamom, coconut, beet, flaxseed, oat, pea or any combination thereof.

9. The composition of claim 1, further comprising a nutrient.

10. The composition of claim 9, wherein the nutrient is zinc, sodium, chloride, citrate, calcium, magnesium, folate, bismuth, vitamin A, a B vitamin, vitamin C, vitamin D, vitamin E, vitamin K2, iron, selenium, manganese, silica, potassium, sulfur, copper, choline, a fatty acid, or any combination thereof.

11. The composition of claim 1, further comprising a sweetener.

12. The composition of claim 11, wherein the sweetener is honey, syrup, chocolate, molasses, fruit, cane sugar, saccharin, sucralose, xylitol or any combination thereof.

13. The composition of claim 1, further comprising a prebiotic, a probiotic, menthol, collagen, hydroxyapatite, fluoride, xanthan gum, dicalcium phosphate, magnesium stearate, an oil, vegetable glycerin, cellulose, gelatin, pea protein, lecithin, mushroom, fungal mycelium, cannabinoids, glucosamine, chondroitin, aloe vera, resin, balsam, sap, wax, fat, beeswax, ozone, or any combination thereof.

14. The composition of claim 1, formulated in water, saline, oil, a gel, a balm, wax, saline, paste, a dissolving strip/wafer, a candy, a gummy, a lozenge, a suppository, a tablet, or a capsule.

15. A composition comprising a derivative of microalgae (e.g. extract, fraction, isolate), formulated for ingestion, application to skin, an oral cavity, or administration rectally to a mammal.

16. The composition of claim 15, at a concentration of 10 mg/ml or less.

17. The composition of claim 15, formulated in water, saline, oil, a gel, a balm, wax, saline, paste, a dissolving strip/wafer, a candy, a gummy, a lozenge, a suppository, an enema, a tablet, or a capsule.

18. A method of treating a mammal, the method comprising administering the composition of any one of claims 1-17 to the mammal.

19. The method of claim 18, wherein the composition is administered orally to the mammal.

20. The method of claim 18, wherein the composition is administered to skin of the mammal.

21. The method of claim 18, wherein the composition is administered to an oral cavity of the mammal.

22. The method of claim 18, wherein the composition is administered rectally to the mammal.

23. The method of claim 18, wherein the mammal is a human.

24. The method of claim 18, wherein the mammal is a dog, cat, rodent, horse, pig, cow, sheep, goat, llama, alpaca, buffalo, camel, ram, donkey, rabbit, yak, mule, bison or ox.
